(19) United States
(12) Patent Application Publication
(10) Pub. No.: US 2016/0151165 A1
(43) Pub. Date: Jun. 2, 2016

(54) CORRECTION OF FIRST RAY DEFORMITY

(71) Applicant: First Ray, LLC, Logan, UT (US)

(72) Inventors: T. Wade Fallin, Hyde Park, UT (US); Robert W. Hoy, Essex Junction, VT (US)

(21) Appl. No.: 14/947,665

(22) Filed: Nov. 20, 2015

Related U.S. Application Data
(60) Provisional application No. 62/085,769, filed on Dec. 1, 2014.
(60) Provisional application No. 62/086,589, filed on Dec. 2, 2014.

(51) Int. Cl. A61F 2/42; A61F 2/08

(52) U.S. Cl. CPC A61F 2/4225; A61F 2/08; A61F 2002/4233

(57) ABSTRACT

Implants and techniques for correcting deformity of the first ray of a human foot are presented. The correction includes realigning and stabilizing the metatarsophalangeal and/or metatarsocuneiform joints of the first ray of the human foot.

### CROSS-REFERENCE TO RELATED APPLICATIONS

This application claims the benefit of U.S. Provisional Application No. 62/085,769, filed Dec. 1, 2014 and U.S. Provisional Application No. 62/086,589, filed Dec. 2, 2014.

### FIELD OF THE INVENTION

The invention relates to methods, implants, and instruments for correcting first ray deformity at the metatarsophalangeal joint and/or metatarsocuneiform joint of the human foot.

### BACKGROUND

Various conditions may affect skeletal joints such as the elongation, shortening, or rupture of soft tissues associated with the joint and consequent laxity, pain, and/or deformity. Repairs of the soft tissues of joints such as those found in the human foot have been difficult. Effective, long lasting correction of deformities of the first ray of the human foot are needed.

### SUMMARY

The present invention provides methods, implants, and instruments for correcting first ray deformity at the metatarsophalangeal joint and/or metatarsocuneiform joint of the human foot.

In one example of the invention, a method of correcting a deformity of the first ray of the human foot includes reducing the metatarsophalangeal joint to at least partially correct the deformity by changing the relative position of the first metatarsus and proximal phalanx to place them in a reduced position; and stabilizing the metatarsophalangeal joint by securing or augmenting soft tissue adjacent to the metatarsophalangeal joint.

In another example of the invention, a graft operable to stabilize a metatarsophalangeal joint includes a generally planar structure having a generally triangular or trapezoidal shape, the base and height of the graft being sized to span a metatarsophalangeal joint of a first ray of a human foot.

### BRIEF DESCRIPTION OF THE DRAWINGS

Various examples of the present invention will be discussed with reference to the appended drawings. These drawings depict only illustrative examples of the invention and are not to be considered limiting of its scope.

FIG. 1 is side elevation view of a foot illustrating anatomic reference planes and relative directions;

FIG. 2 is a lateral view of a foot illustrating dorsiflexion and plantar flexion;

FIG. 3 is a coronal view of a foot illustrating inversion and eversion;

FIG. 4 is a dorsal view illustrating bones, tendons, and ligaments of the foot;

FIG. 5 is a plantar view illustrating bones, tendons, and ligaments of the foot;

FIG. 6 is a perspective view illustrating bones, tendons, and ligaments of the foot;

FIG. 7 is a medial view of the MTP joint of the first ray of the foot;

FIG. 8 is a sectional view taken along line 8-8 of FIG. 7;

FIG. 9 is a dorsal view of the MTC joint of the first ray of the foot;

FIG. 10 is a medial view of the MTC joint of the first ray of the foot;

FIG. 11 is a dorsal view illustrating deformity of the foot;

FIG. 12 is a plantar view illustrating deformity of the foot;

FIG. 13 is a sectional view similar to that of FIG. 8 but illustrating deformity of the foot;

FIGS. 14-20 are medial views of the MTP joint undergoing correction of a deformity; and

FIGS. 21-27 are dorsal and medial views of the MTC joint undergoing correction of a deformity.

### DESCRIPTION OF THE ILLUSTRATIVE EXAMPLES

The following illustrative examples describe implants, instruments and techniques for treating deformity of the first ray of the human foot. In particular, they describe ways of treating hallux valgus by correcting soft tissue deficiencies relating to the metatarsophalangeal joint of the first ray.

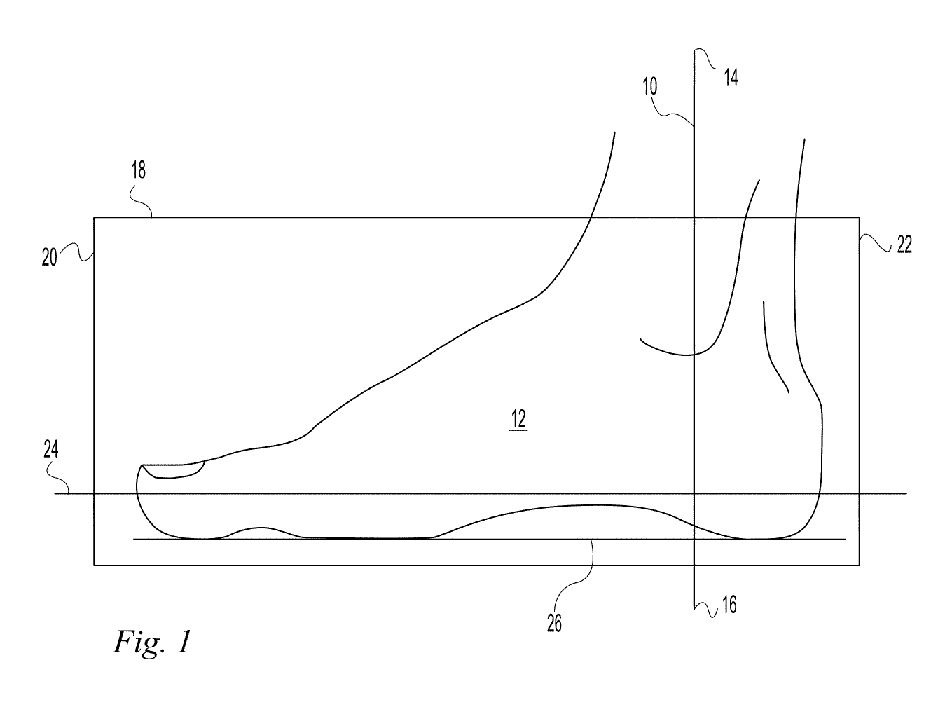

FIG. 1 illustrates the orientation of anatomic planes and relative directional terms that are used for reference in this application. The coronal plane 10 extends from medial 12 (toward the midline of the body) to lateral (away from the midline of the body) and from dorsal 14 (toward the top of the foot) to plantar 16 (toward the sole of the foot). The sagittal plane 18 extends from anterior 20 (toward the front of the body) to posterior 22 (toward the back of the body) and from dorsal 14 to plantar 16. The transverse plane 24 extends anterior 20 to posterior 22 and medial to lateral parallel to the floor 26. Relative positions are also described as being proximal or distal where proximal is along the lower extremity toward the knee and distal is along the lower extremity toward the toes. The following examples serve to demonstrate the relative directions. The great toe is medial of the lesser toes and the fifth toe is lateral of the great toe. The toes are distal to the heel and the ankle is proximal to the toes. The instep is dorsal and the arch is plantar. The toenails are dorsal and distal on the toes.

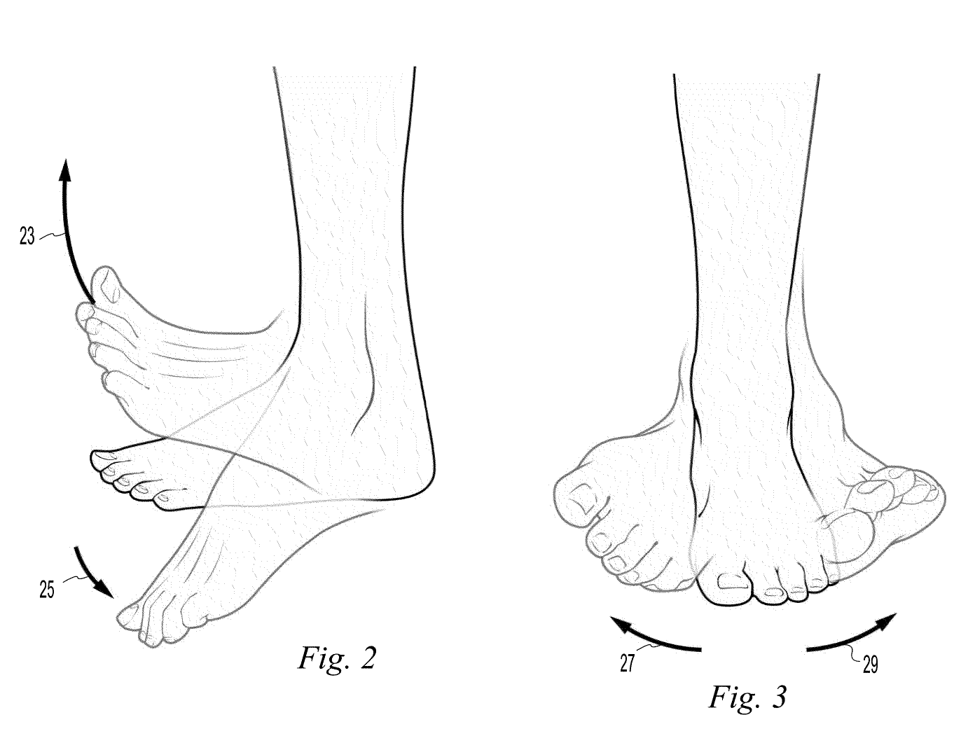

FIG. 2 illustrates dorsiflexion 23 in which the toes are moved dorsally, or closer to the shin, by decreasing the angle between the dorsum of the foot and the leg and plantar flexion 25 in which the toes are moved plantarly, or further away from the shin, by increasing the angle between the dorsum of the foot and the leg. For example when one walks on their heels, the ankle is dorsiflexed and when one walks on their toes, the ankle is plantar flexed.

FIG. 3 illustrates inversion 27 in which the sole of the foot is tilted toward the sagittal plane or midline of the body and eversion 29 in which the sole of the foot is tilted away from the sagittal plane.

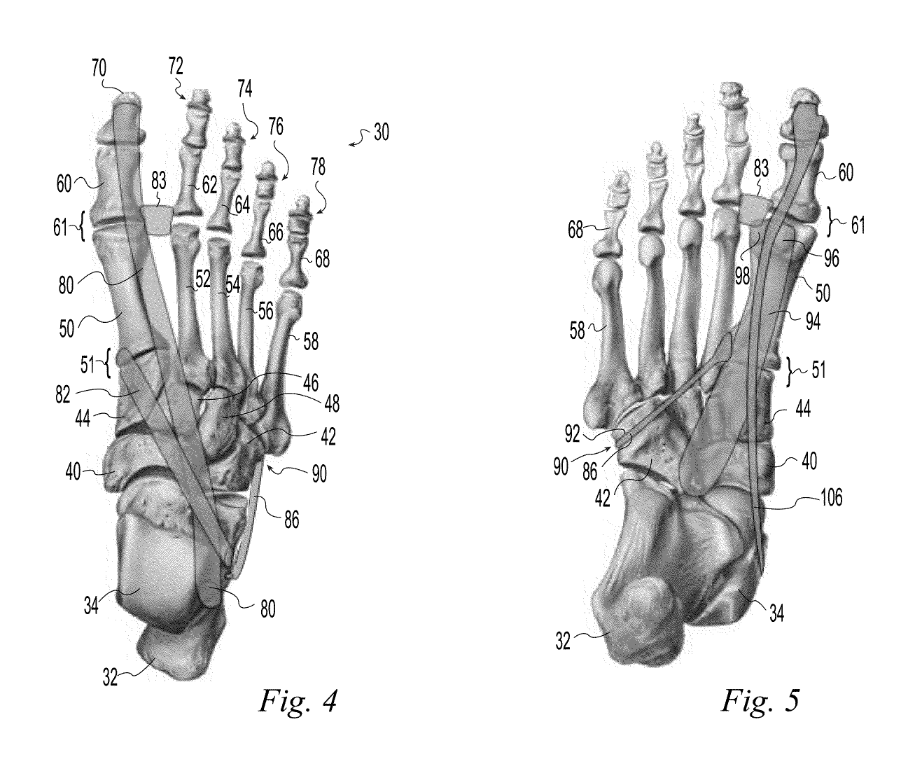

FIGS. 4-10 illustrate the arrangement of the bones within the foot 30. A right foot is illustrated. Beginning at the proximal aspect of the foot, the heel bone or calcaneus 32 projects plantar. The talus 34 is dorsal to the calcaneus 32 and articulates with it at the talocalcaneal or subtalar joint. Dorsally, the talus articulates medially with the tibia 36 and laterally with the fibula 38 at the ankle joint. Distal to the ankle are the navicular bone 40 medially and the cuboid bone 42 laterally which articulate with the talus and calcaneus respectively. The navicular bone 40 and cuboid bone 42 may also articulate with one another at the lateral side of the navicular bone and the medial side of the cuboid bone. Three cuneiform bones lie distal to the navicular bone and articulate with the navicular bone and one another. The first, or medial, cuneiform 44 is located on the medial side of the foot 30. The second, or intermediate, cuneiform 46 is located lateral of the first cuneiform 44. The third, or lateral, cuneiform 48 is located lateral of the second cuneiform 46. The third cuneiform 48 also articulates with the cuboid bone 42. Five metatarsals 50, 52, 54, 56, 58 extend distally from and articulate with the cuneiform and cuboid bones. The metatarsals are numbered from 1 to 5 starting with the first metatarsal 50 on the medial side of the foot and ending with the fifth metatarsal 58 on the lateral side of the foot 30. The first metatarsal 50 articulates with the first cuneiform 44 at a metatarsocuneiform (MTC) joint 51. The second metatarsal 52 articulates with the first, second and third cuneiforms 44, 46, 48. Five proximal phalanges 60, 62, 64, 66, 68 extend distally from and articulate with the five metatarsals respectively. The first proximal phalanx 60 articulates with the first metatarsal 50 at a metatarsophalangeal (MTP) joint 61. One or more distal phalanges 70, 72, 74, 76, 78 extend distally from the proximal phalanges. The first metatarsal 50, first proximal phalanx 60, and, first distal phalanx 70 together are referred to as the first ray of the foot. Similarly, the metatarsal, proximal phalanx, and distal phalanges corresponding to the lesser digits are referred to as the second through fifth rays respectively.

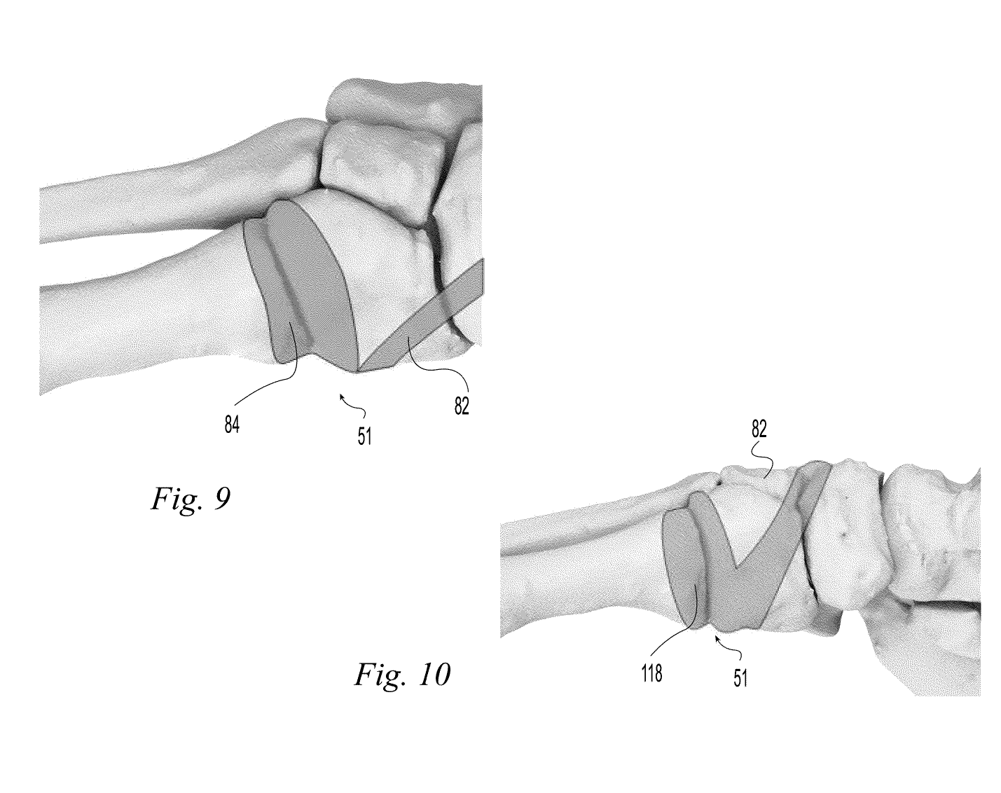

FIG. 4 is a dorsal view illustrating bones, tendons and ligaments of the foot. Plantar structures illustrated in FIG. 5 are omitted from FIG. 4 for clarity. The extensor hallucis longus muscle originates in the anterior portion of the leg, the extensor hallucis longus tendon 80 extends distally across the ankle and along the first ray to insert into the base of the distal phalanx 70. The tibialis anterior muscle originates in the lateral portion of the leg and the tibialis anterior tendon 82 extends distally across the ankle and inserts into the first cuneiform 44 and first metatarsus 50 at the first MTC joint 51 where it contributes to the MTC capsular structure 84 (FIG. 9). A transverse intermetatarsal ligament 83 connects the heads of the first through fifth metatarsal bones. In FIG. 4, only the connection between the first and second metatarsal bones 50, 52 is shown. The intermetatarsal ligament inserts into the capsule of the MTP joint.

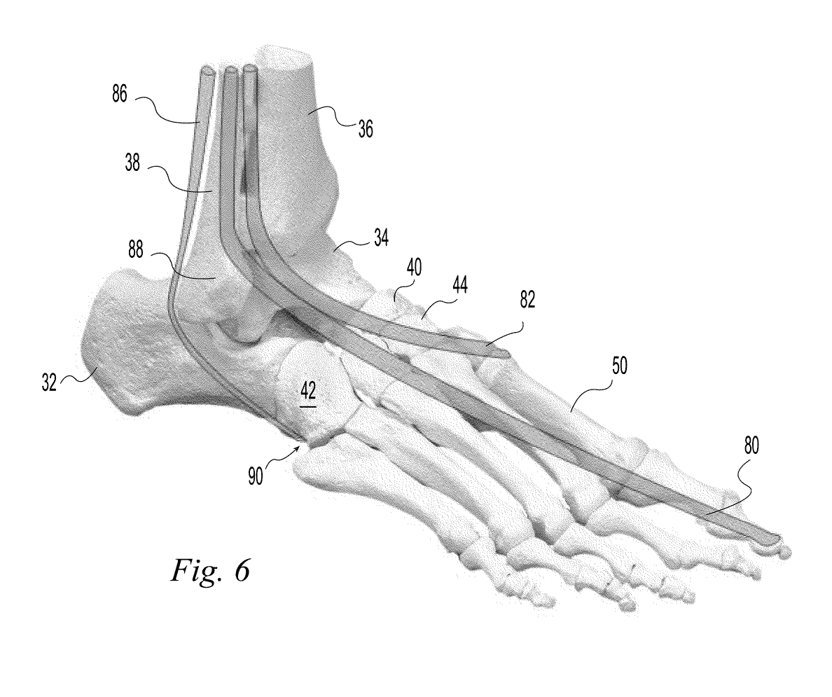

FIG. 5 is a plantar view illustrating bones, tendons, and ligaments of the foot. Dorsal structures shown in FIG. 4 are omitted from FIG. 5 for clarity. The peroneus longus muscle originates at the head of the fibula and its tendon 86 tendon passes posteriorly around the lateral malleolus 88 (FIG. 6) of the ankle, around the cuboid notch 90 on the lateral side of the cuboid bone 42, along the peroneus sulcus 92 on the plantar surface of the cuboid bone 42, and inserts into the first metatarsal 50. The flexor hallucis brevis muscle 94 originates from the cuboid 42 and third cuneiform 48 and divides distally where it inserts into the base of the proximal phalanx 60. Medial and lateral sesamoid bones 96, 98 are present in each portion of the divided tendon at the MTP joint 61. The sesamoids 96, 98 articulate with the planar surface of the metatarsal head in two grooves 100, 102 separated by a rounded ridge, or crista 104 (FIG. 8). The flexor hallucis longus muscle originates from the posterior portion of the fibula 38. The flexor hallucis longus tendon 106 crosses the posterior surface of the lower end of the tibia, the posterior surface of the talus, runs forward between the two heads of the flexor hallucis brevis 94, and is inserted into the base of the distal phalanx 70 of the great toe.

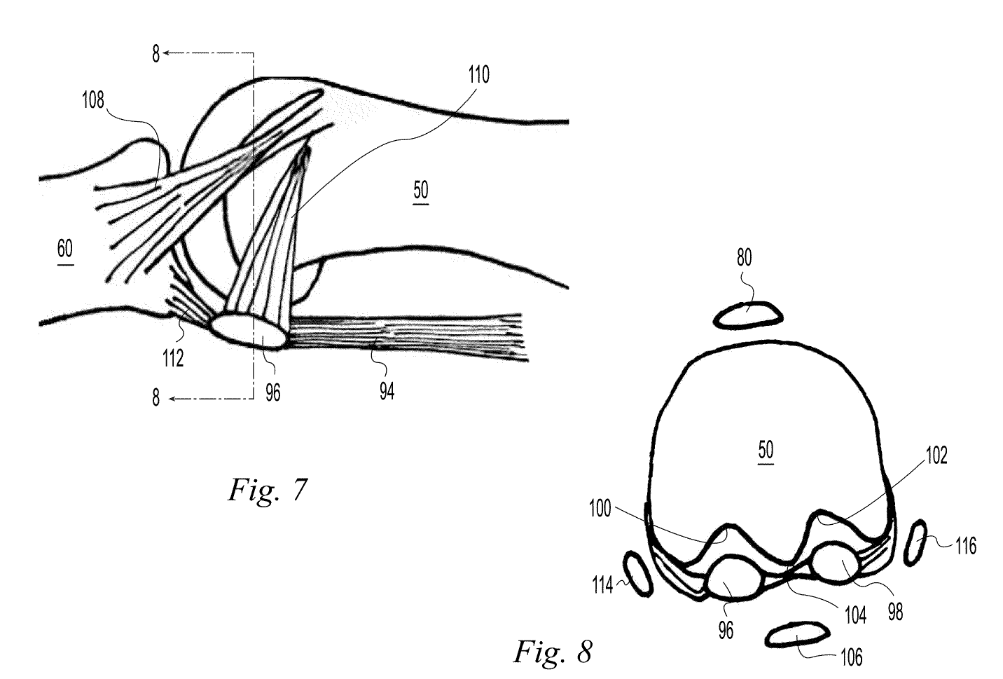

FIG. 7 is a medial view of tendons at the MTP joint 61 of the first ray. A medial collateral ligament 108 originates from the head of the first metatarsus 50 and inserts into the proximal phalanx 60. A medial metatarsosesamoid ligament 110 originates from the head of the first metatarsus 50 and inserts into the medial sesamoid bone 96. Similar collateral and metatarsosesamoid ligaments are found on the medial side of the first MTP joint. The flexor hallucis brevis 94 is shown inserting into the sesamoids 96, 98. Ligamentous fibers extend further distally in the form of a phalangealsesamoid ligament 112 from the sesamoids to the proximal phalanx 60.

FIG. 8 is a sectional view taken along line 8-8 of FIG. 7 showing the metatarsal head 50, the tendon of the extensor hallucis longus 80, the medial and lateral sesamoid bones 96, 98, the grooves 100, 102 in which the sesamoids articulate, the crista 104 separating the grooves, the flexor hallucis longus 106, the abductor hallucis 114, and the adductor hallucis 116.

FIG. 9 is a dorsal view showing the dorsal capsular structure 84 of the MTC joint 51 of the first ray including the insertion of the tibialis anterior tendon 82.

FIG. 10 is a medial view of the MTC joint 51 of the first ray showing the medial capsular structure 118.

FIGS. 11-13 illustrate deformities of the first ray. In a dorsal view, as shown in FIG. 11, an intermetatarsal angle (IMA) 120 may be measured between the longitudinal axes of the first and second metatarsal bones 50, 52. The angle is considered abnormal when it is 9 degrees or greater and the condition is known as metatarsus primus varus (MPV) deformity. A mild deformity is less than 12 degrees, a moderate deformity is 12-15 degrees, and a severe deformity is greater than 15 degrees. Similarly, a hallux valgus angle (HVA) 122 may be measured between the longitudinal axes of the first metatarsus 50 and the first proximal phalanx 60 at the MTP joint 61. The angle is considered abnormal when it is 15 degrees or greater and the condition is known as a hallux valgus (HV) deformity. A mild deformity is less than 20 degrees, a moderate deformity is 20 to 40 degrees, and a severe deformity is greater than 40 degrees.

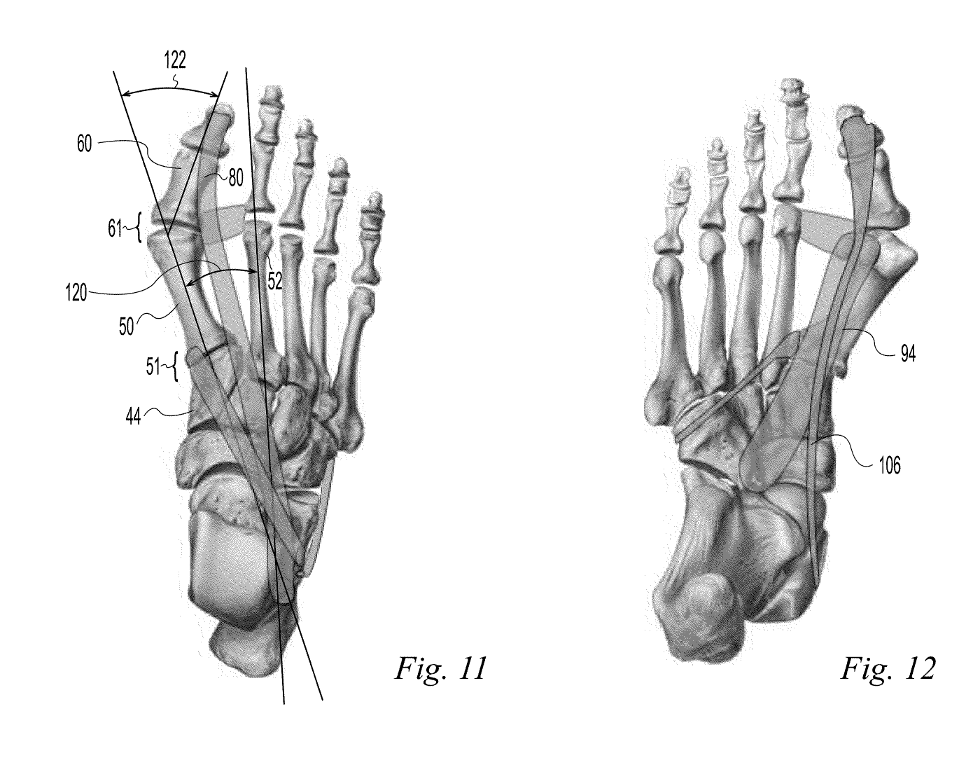

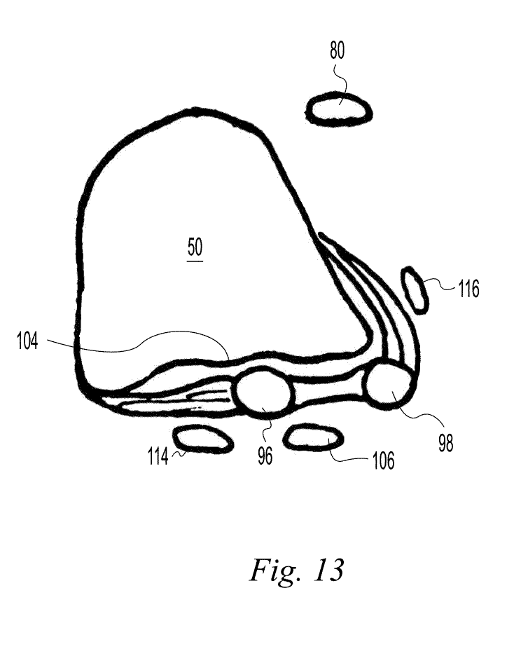

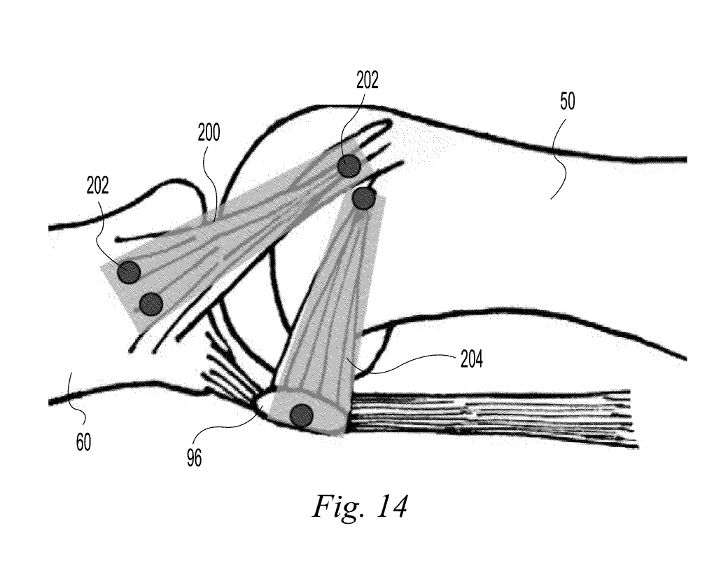

MPV and HV often occur together as shown in FIGS. 11-12. As the deformities progress several changes may occur in and around the MTC and MTP joints. Referring to FIG. 13, as the IMA and HVA increase, the extensors 80, flexors 106, abductors 114, and adductors 116 of the first ray (along with the sesamoids 96, 98) are shifted laterally relative to the MTP joint. The laterally shifted tendons exert tension lateral to the MTP joint creating a bow string effect (as best seen in FIGS. 11 and 12) that tends to cause the deformities to increase. The lateral shift of the sesamoids 96, 98 is often accompanied by erosion of the crista. The abnormal muscle forces cause the metatarsus 50 to pronate, or in other words, rotate so that the dorsal aspect of the bone moves medially and the plantar aspect moves laterally. Rotation in the opposite direction is referred to as supination. Soft tissues on the medial side of the MTP joint and lateral side of the MTC joint attenuate, through lengthening and thinning, thus weakening the capsule and permitting the deformities to progress. Soft tissues on the opposite sides of the capsule tend to shorten, thicken and form contractures making it difficult to reduce the joints to their normal angular alignment.

More generally, deformities of the first ray may include metatarsus primus varus, hallux valgus, abnormal pronation, abnormal supination, abnormal dorsiflexion, and/or abnormal plantarflexion. These deformities correspond to three different planar rotations. Metatarsus primus varus and hallux valgus result from rotations in the transverse plane 24. Pronation and supination are rotation in the coronal plane 10. Dorsiflexion and plantar flexion are rotation in the sagittal plane 20.

The terms “suture” and “suture strand” are used herein to mean any strand or flexible member, natural or synthetic, able to be passed through material and useful in a surgical procedure. The term “transverse” is used herein to mean crossing as in non-parallel.

According to the present invention first ray deformity may be corrected at one or both of the MTP or MTC joints. For example, referring to the MTP joint, the first metatarsus and proximal phalanx bones 50, 60 may be relatively rotated in one or more planes to an abnormal position. The deformity may be corrected by reducing the MTP joint to at least partially correct the deformity by changing the relative position of the first metatarsus and proximal phalanx to place them in a reduced position and then stabilizing the MTP joint by securing and/or augmenting soft tissue adjacent to the MTP joint. For example, in HV deformity, the first metatarsus and proximal phalanx bones 50, 60 may be relatively rotated in the transverse plane to reduce the hallux valgus angle and the joint then stabilized. The correction may involve relative rotation of the bones in more than one plane. For example the proximal phalanx may be abnormally pronated or supinated relative to the first metatarsus. The correction according to the present invention may include relative rotation of the bones in the coronal plane to correct abnormal pronation or supination prior to stabilizing the joint. Likewise, it may be desirable to relatively rotate the bones in the sagittal plane to correct abnormal dorsiflexion or plantar flexion. Correction according to the present invention may be uni-planar, bi-planar, or tri-planar.

In addition to correcting the angular deformity, a portion of the bone of the metatarsal head may be removed in a procedure known as an exostectomy or bunionectomy to reduce medial prominence. As part of a bunionectomy, the capsular tissue overlying the bone portion to be removed is dissected out of the way. The present invention provides for restoring the function of the capsular tissue so dissected.

According to various examples of the present invention, the MTP joint may be hyper mobile and need stabilization in a corrected position. Alternatively, surgical access to the MTP joint may cause the joint to become sufficiently mobile to allow reduction of the joint to a desired position. However, it is likely that the method according to the present invention will require freeing soft tissue that restricts motion of the MTP joint in order to permit reduction of the joint. For example it may be necessary to free contractures by resecting or lengthening the contracted tissue. For example, it may be necessary to free contractures in the joint capsule and/or in ligaments or tendons that attach to the first metatarsus 50, proximal phalanx 60 or sesamoid bones 96,98. For example, this may include the capsule proper as well as collateral ligaments, metatarsosesamoid ligaments, transverse metatarsal ligaments, adductor tendons and other structures that originate or insert near the joint.

The MTP joint may be stabilized by securing soft tissue adjacent to the joint using various techniques. For example, direct fixation of the capsular tissue may be carried out using screws, staples, tacks, nails, suture anchors and/or sutures to tighten the capsule or redirect forces within the capsular tissue. Sutures may be placed between soft tissues, anchored intraosseously, and/or anchored with suture anchors. MTP joint capsular tissue may also be detached, moved, and reattached. For example a boney insertion of the capsular tissue may be moved. The MTP joint may be stabilized by transferring a remote soft tissue structure to the metatarsocuneiform joint and fixing the transferred soft tissue. For example, a tendon or ligament from another part of the foot may be detached and transferred to the MTP joint where it may be attached to other soft tissues, the first metatarsus, the proximal phalanx, and/or one or more sesamoid bones.

In another illustrative example of the present invention, the MTP joint may be stabilized by attaching a graft at the MTP joint. For example a graft may be attached from one portion of the capsule or other soft tissue to another to strengthen absent or attenuated tissues. A graft may be attached in one or more locations to underlying bone. The graft may be attached to one or more bones. For example the graft may be attached to one or more of the first metatarsus, proximal phalanx, and sesamoid bones. The graft may be attached to a bone and a soft tissue.

A graft according to the illustrative example of the present invention may be a scaffold or a frank replacement and it may be synthetic or natural. Natural grafts may be autograft, allograft, or xenograft. The graft may be attached with screws, staples, tacks, nails, suture anchors and/or sutures. The graft may be a unitary structure that augments or replaces the stabilizing action of a single portion of the capsule or other soft tissue. The graft may be a unitary structure that augments or replaces the stabilizing action of a plurality of capsular ligaments or other soft tissues. The graft may include a plurality of discrete structures that augment or replace the stabilizing action of a plurality of capsular ligaments or other soft tissues.

FIG. 14 illustrates MTP joint stabilization with a graft 200. In the illustrative example of FIG. 14, stabilization is shown on the medial side of the joint as would be typical for stabilizing a reduced hallux valgus deformity. However, a similar graft may be placed on the lateral side of the joint if lateral stabilization is desired. In the illustrative example of FIG. 14, anchors 202 have been placed in the first metatarsus 50 and proximal phalanx 60 near the MTP joint to secure the graft 200. The graft 200 is sized to span the origin and insertion of the collateral ligament of the MTP joint. In the illustrative example of FIG. 14, a second graft 204 is similarly attached between the first metatarsus 50 and medial sesamoid bone 96 and is sized to span the origin and insertion of the metatarsosesamoid ligament.

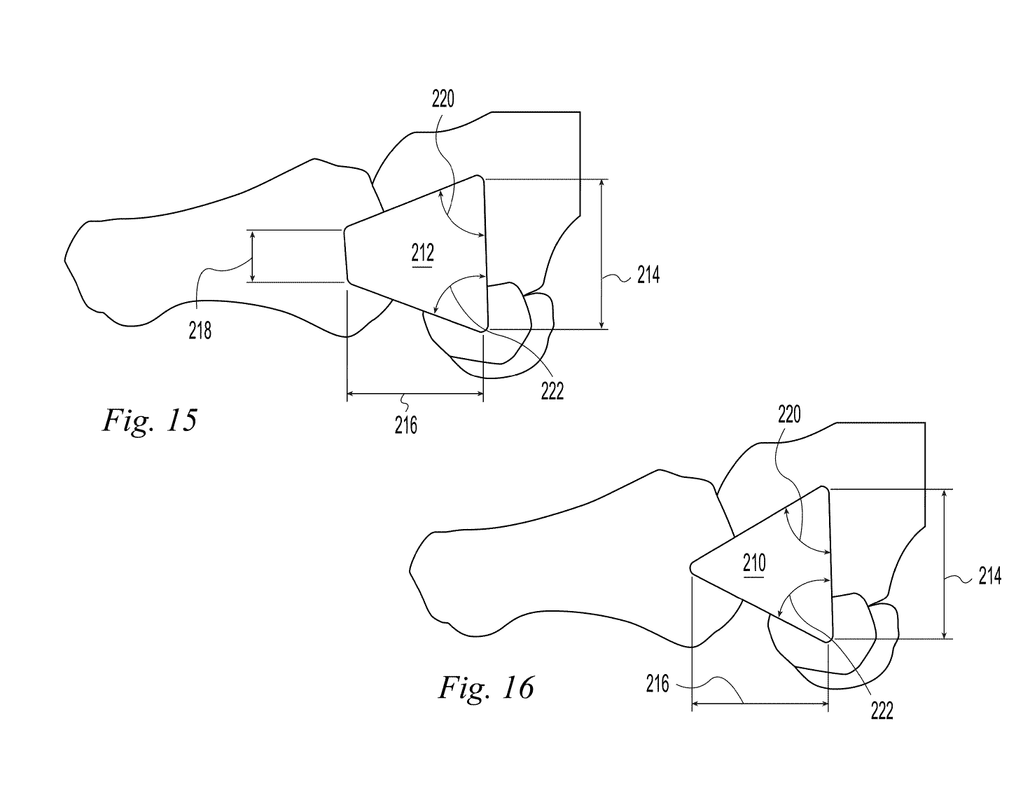

FIGS. 15 and 16 illustrate example alternative graft footprints for the MTP joint. For example a graft may have three or more vertices with a base dimension defined between two of the vertices and a height dimension from the base dimension to a third vertex normal to the base dimension. For example, a graft may be generally triangular 210 as shown in FIG. 16 or generally trapezoidal 212 as shown in FIG. 15. In the illustrative examples of FIGS. 15 and 16, the graft has a base dimension 214 in the range of 11-35 mm and a height dimension 216 in the range of 9-29 mm. More particularly the graft has a base dimension in the range of 18-28 mm and a height in the range of 14-23 mm. More particularly the graft has a base dimension in the range of 20-26 mm and a height in the range of 17-21 mm. In the case of a trapezoidal graft, the graft has a second base dimension 218 in the range of 4-14 mm; more particularly 7-11 mm, and still more particularly 7-10 mm.

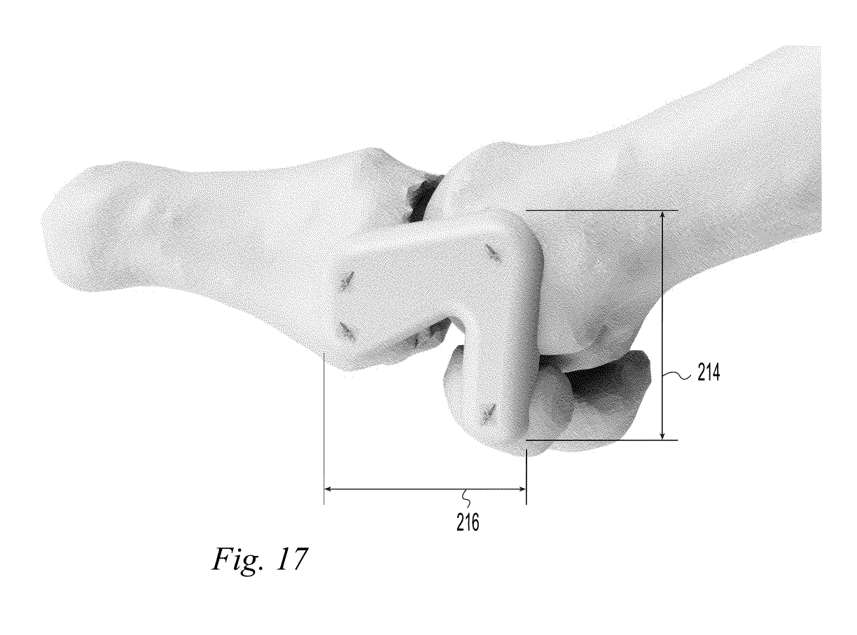

In the illustrative examples of FIGS. 15 and 16, the grafts are shown as regular polygons with base angles 220 and 222 that are equal. However, the angles may be unequal. Likewise, in the illustrative examples of FIGS. 15 and 16, the grafts are shown with rounded vertices. However, the vertices may come to a sharp point or some other shape. Likewise, while the illustrative grafts of FIGS. 15 and 16 have been depicted as a smooth sided triangle and a smooth sided trapezoid, the grafts may be of any shape between the vertices. For example, the sides may be curved, convex, concave, lobed, notched, irregular, or any other shape. For example, a side may have a concave curve such that the graft takes on a “Y” shape while still being within the example base and height ranges as shown in FIG. 17.

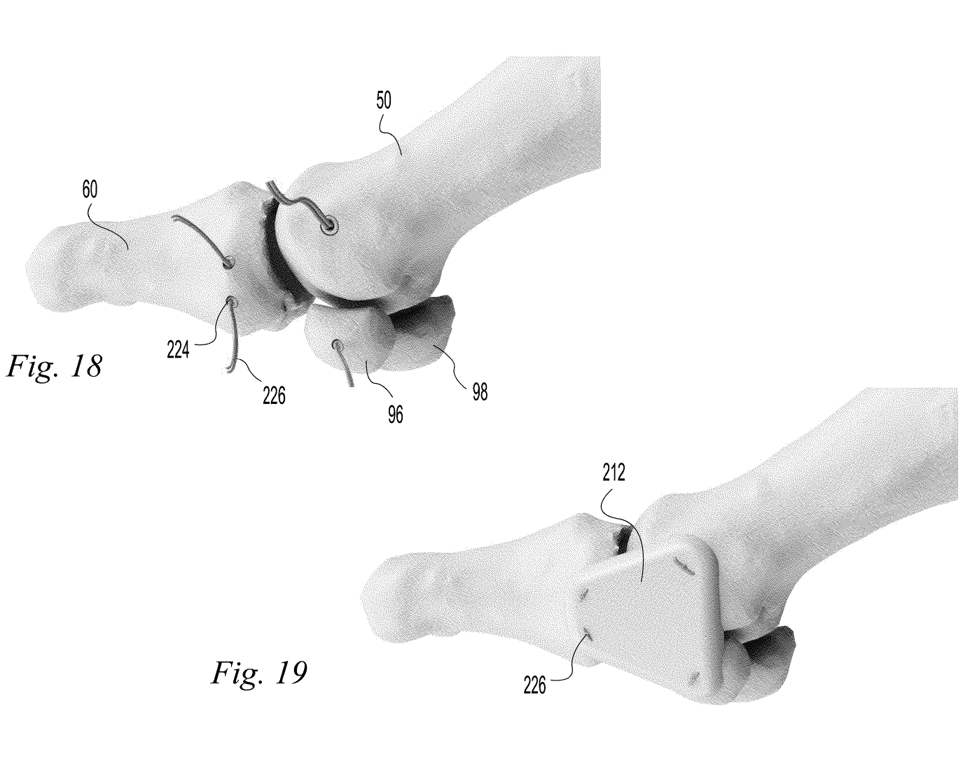

FIGS. 18 and 19 illustrate MTP joint stabilization utilizing the trapezoidal graft 212 of FIG. 15 on the medial side of the MTP joint. In the illustrative example of FIGS. 18 and 19, suture anchors 224 have been placed in the first metatarsus 50, proximal phalanx 60, and medial sesamoid 96 near the MTP joint. Preferably, the suture anchors 224 are placed at the origins and insertions of the medial collateral ligament and medial metatarsosesamoid ligament. Sutures 226 extending from the anchors 224 are passed through the graft 212 and used to secure it to the bones.

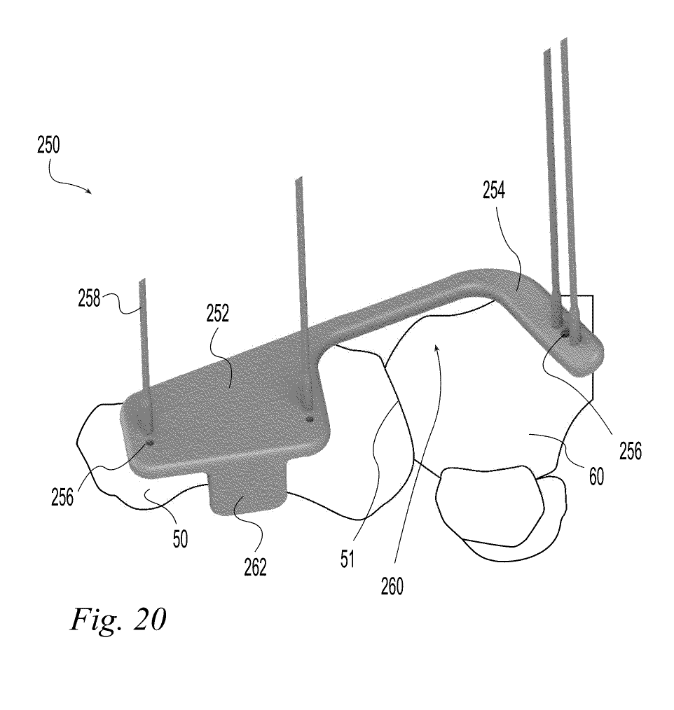

FIG. 20 illustrates a temporary fixation instrument 250 for temporarily securing the first metatarsus 50 and proximal phalanx 60 while the MTP joint is stabilized. The instrument 250 includes a first portion 252 for attachment to the first metatarsus 50 and a second portion 254 for attachment to the proximal phalanx 60. The first and second portions 252, 254 are rigidly connected. In the illustrative example of FIG. 20, the first and second portions 252, 254 are a unitary structure. Alternatively, the first and second portions 252, 254 may be adjustable relative to one another and then locked in a rigid configuration. In the illustrative example of FIG. 20, the instrument 250 has a generally planar configuration. The first and second portions 252, 254 include through holes 256 for receiving wires 258 for securing the instrument 250 to the bones. A window 260 formed between the first and second portions 252, 254 provides access to the MTP joint. In the illustrative example of FIG. 20, the first portion 252 includes a tab 262 extending from the first portion 252 to aid positioning the instrument 250 on the bones.

In use, the instrument 250 is placed adjacent to the first metatarsus 50 and proximal phalanx 60 and positioned so that at least some of the holes 256 align with each of the bones and the window 260 is aligned with the MTP joint 51. The tab 262 may be placed against the first metatarsus 50 as shown in FIG. 20 to aid in alignment and help stabilize the plate while the wires 258 are inserted. For example, the instrument 250 may be positioned against the medial side of the bones with the tab 262 abutting the plantar surface of the first metatarsus 50. The plate may be stabilized, for example, by placing a finger of the user's hand on the tab 262. The relative position of the first metatarsus 50 and proximal phalanx 60 is adjusted in one or more planes to place the MTP joint in a reduced position to at least partially correct a deformity of the first ray. Wires 258 are inserted through the holes 256 and into the underlying bone to secure the plate to the bones and temporarily fix the bones in the reduced position. The MTP joint is then stabilized by securing or augmenting soft tissue adjacent to the joint such as by one of the previously described joint stabilizing techniques according to the present invention. Access to the joint is facilitated by the window 260. The wires 258 and instrument 250 are then removed.

A first ray deformity may also be corrected by reducing the MTC joint to at least partially correct the deformity by changing the relative position of the first metatarsus and first cuneiform to place them in a reduced position and then stabilizing the MTC joint by securing and/or augmenting soft tissue adjacent to the MTC joint as described relative to the MTP joint above. For example, in MPV deformity, the first metatarsus and first cuneiform bones 50, 44 may be relatively rotated in the transverse plane to reduce the intermetatarsal angle and the joint stabilized. The correction may involve relative rotation of the bones in more than one plane. For example, in MPV deformity, the first metatarsus is sometimes abnormally pronated. The correction according to the present invention may include relative rotation of the bones in the coronal plane to correct abnormal pronation prior to stabilizing the joint. Likewise, it may be desirable to relatively rotate the bones in the sagittal plane to correct abnormal dorsiflexion or plantar flexion. Correction according to the present invention may then be uni-planar, bi-planar, or tri-planar.

As described relative to the MTP joint, the MTC joint may be hyper mobile and need stabilization in a corrected position. Alternatively, surgical access to the MTC joint may cause the joint to be sufficiently mobile to allow reduction of the joint to a desired position. However, it is likely that the method according to the present invention will require freeing soft tissue that restricts motion of the MTC joint in order to permit reduction of the joint. For example it may be necessary to free contractures by resecting or lengthening the contracted tissue. For example, it may be necessary to free contractures in the joint capsule and/or in ligaments or tendons that attach to the first metatarsus or first cuneiform and restrict their relative motion. This may include the capsule proper as well as extensors, flexors, ligaments, and other structures that originate or insert near the joint.

The MTC joint may be stabilized by securing soft tissue adjacent to the joint using the various techniques and fixation described relative to the MTP joint including, for example, stabilizing the MTC joint by attaching a graft at the MTC joint. For example a graft may be attached from one portion of the capsule or other soft tissue to another to strengthen absent or attenuated tissues. A graft may be attached in one or more locations to underlying bone. The graft may be attached to one or more bones. For example the graft may be attached to one or more of the first metatarsus, first cuneiform, second metatarsus, second cuneiform, navicular, or other suitable bones. The graft may be attached to a bone and a soft tissue.

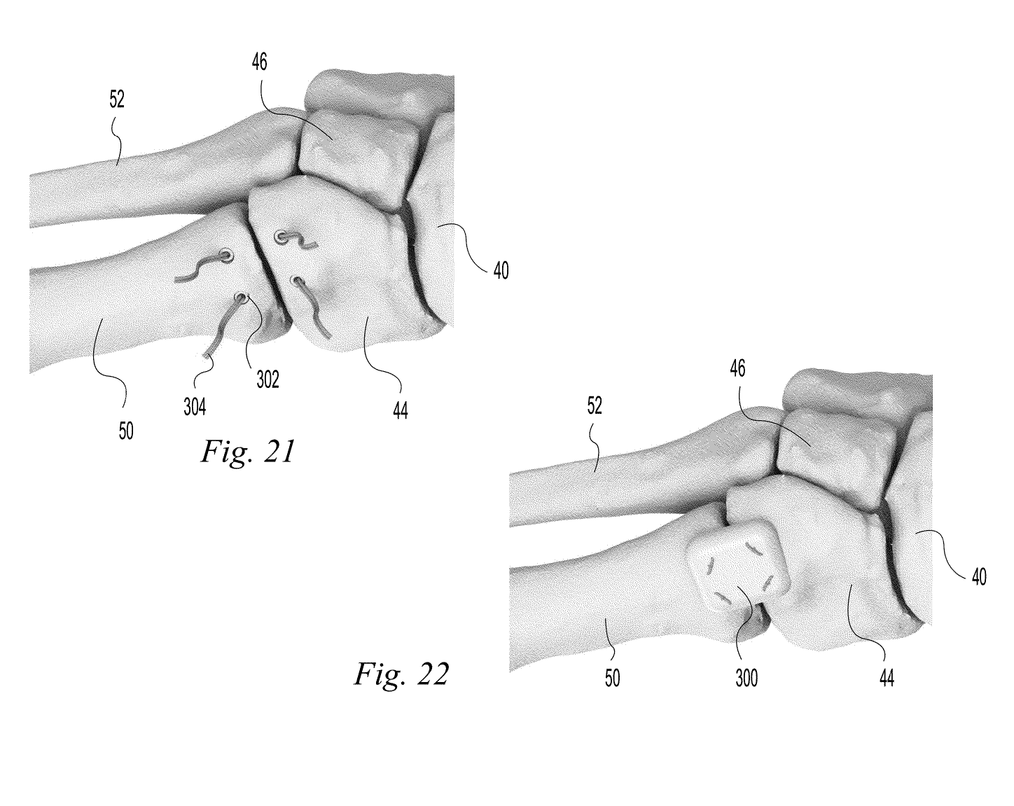

FIGS. 21 and 22 illustrate MTC joint stabilization with a graft 300. In the illustrative example of FIGS. 21 and 22, suture anchors 302 have been placed in the first metatarsus 50 and first cuneiform 44 near the MTC joint. Sutures 304 extending from the anchors 302 are passed through the graft 300 and used to secure it to the bones. In the illustrative example of FIGS. 21 and 22, a relatively small graft is placed dorsally.

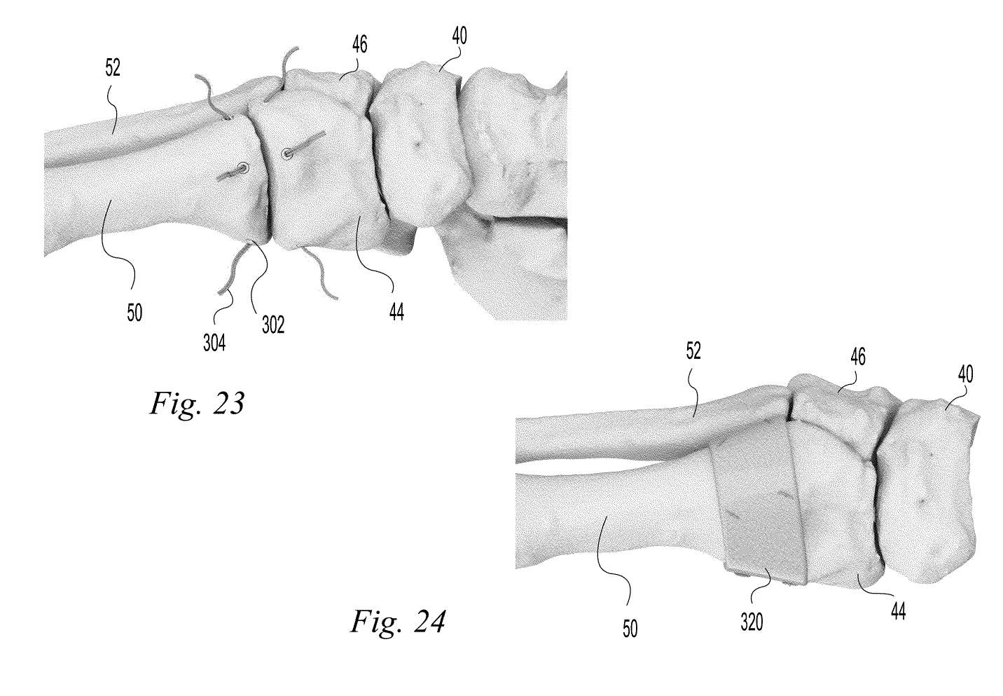

FIGS. 23 and 24 illustrate a graft 320 similar to that of FIGS. 21 and 22 but spanning a larger portion of the MTC joint. Additional suture anchors 302 have been placed and the anchors have been spaced further around the joint. The graft 320 extends from a dorsal portion of the MTC joint to a mediodorsal portion of the joint.

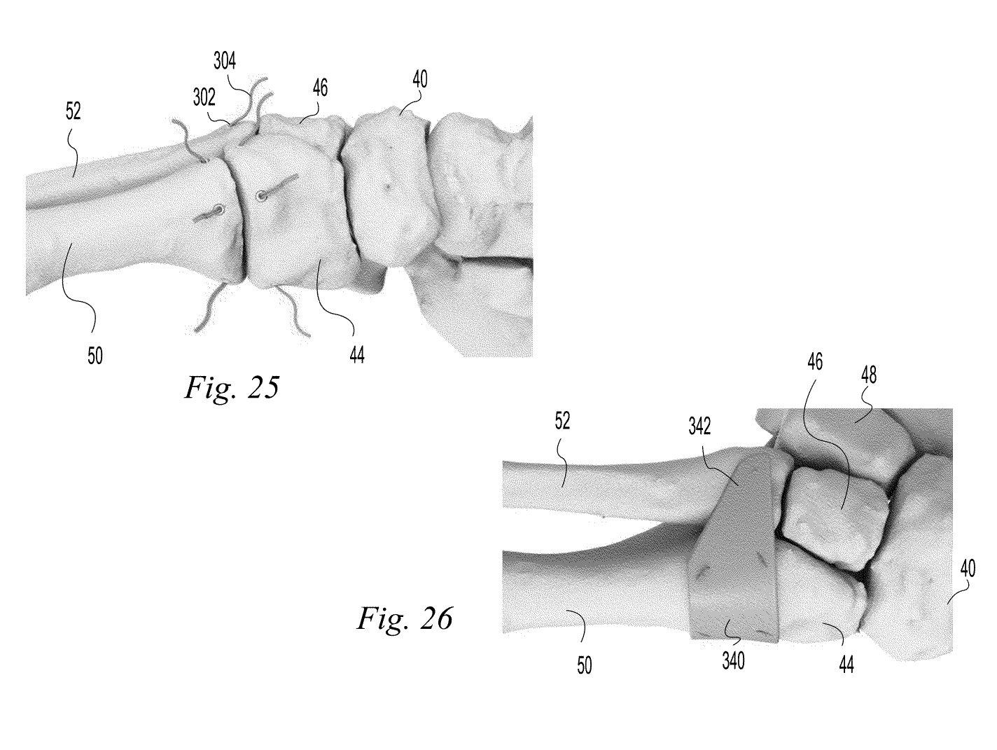

FIGS. 25 and 26 illustrate a graft 340 similar to that of FIGS. 23 and 24 but further including a portion 342 extending to the second metatarsus 52 and attached to the second metatarsus with an addition suture anchor 302 and suture 304 placed in the second metatarsus 52.

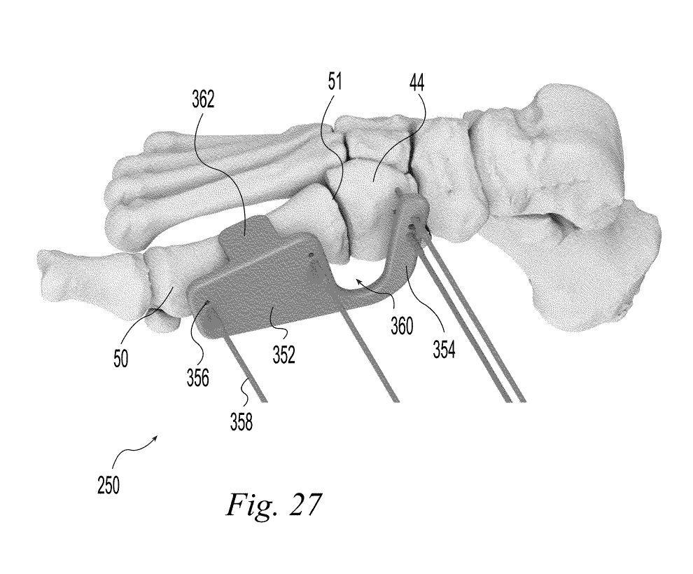

FIG. 27 illustrates a temporary fixation instrument 350 for temporarily securing the first metatarsus 50 and first cuneiform 44 while the MTC joint 51 is stabilized. The instrument 350 includes a first portion 352 for attachment to the first metatarsus 50 and a second portion 354 for attachment to the first cuneiform 44. The first and second portions 352, 354 are rigidly connected. In the illustrative example of FIG. 27, the first and second portions 352, 354 are a unitary structure. Alternatively, the first and second portions 352, 354 may be adjustable relative to one another and then locked in a rigid configuration. In the illustrative example of FIG. 27, the instrument 350 has a generally planar configuration. The first and second portions 352, 354 include through holes 356 for receiving wires 358 for securing the instrument 350 to the bones. A window 360 formed between the first and second portions 352, 354 provides access to the MTC joint 51. In the illustrative example of FIG. 27, the first portion 352 includes a tab 362 extending from the first portion 252 to aid positioning the instrument 350 on the bones.

In use, the instrument 350 is placed adjacent to the first metatarsus 50 and first cuneiform 44 and positioned so that at least some of the holes 356 align with each of the bones and the window 360 is aligned with the MTC joint 51. The tab 362 may be placed against the first metatarsus 50 as shown in FIG. 27 to aid in alignment and help stabilize the plate while the wires 358 are inserted. For example, the instrument 350 may be positioned against the medial side of the bones with the tab 362 abutting the dorsal surface of the first metatarsus 50. The plate may be stabilized, for example, by placing a finger of the user's hand on the tab 362. The relative position of the first metatarsus 50 and first cuneiform 44 is adjusted in one or more planes to place the MTC joint in a reduced position to at least partially correct a deformity of the first ray. Wires 358 are inserted through the holes 356 and into the underlying bone to secure the plate to the bones and temporarily fix the bones in the reduced position. The MTC joint is then stabilized by securing or augmenting soft tissue adjacent to the joint such as by one of the previously described joint stabilizing techniques according to the present invention. Access to the joint is facilitated by the window 360. The wires 358 and instrument 350 are then removed.

The illustrative examples have described instruments, implants and methods for correcting deformity of the first ray of a human foot. The correction includes realigning and stabilizing the MTP joint and/or MTC joint of the first ray. Variations in angular correction, instruments, implants, attachments, and other aspects of the invention have been described in the examples. Combinations of and substitutions among these variations are within the scope of the invention.

What is claimed is:

1. A method of correcting a deformity of the first ray of the human foot, the first ray including a metatarsophalangeal joint between a first metatarsus and a proximal phalanx, the method comprising:
reducing the metatarsophalangeal joint to at least partially correct the deformity by changing the relative position of the first metatarsus and proximal phalanx to place them in a reduced position; and
attaching a graft at the metatarsophalangeal joint to stabilize the metatarsophalangeal joint.

2. The method of claim 1 further comprising before reducing the metatarsophalangeal joint, freeing soft tissue that restricts motion of the metatarsophalangeal joint.

3. The method of claim 1 wherein reducing the metatarsophalangeal joint comprises relative rotation of the first metatarsus and proximal phalanx in a transverse plane.

4. The method of claim 3 wherein relative rotation of the first metatarsus and proximal phalanx in a transverse plane comprises reducing the hallux valgus angle to correct hallux valgus.

5. The method of claim 1 wherein reducing the metatarsophalangeal joint comprises relative rotation of the first metatarsus and proximal phalanx in a coronal plane.

6. The method of claim 5 wherein relative rotation of the first metatarsus and proximal phalanx in a coronal plane comprises reducing abnormal pronation of the proximal phalanx.

7. The method of claim 1 wherein reducing the metatarsophalangeal joint comprises relative rotation of the first metatarsus and proximal phalanx in a sagittal plane.

8. The method of claim 1 wherein reducing the metatarsophalangeal joint comprises relative rotation of the first metatarsus and proximal phalanx in a transverse plane, a coronal plane, and a sagittal plane to achieve a tri-planar reduction.

9. The method of claim 1 further comprising temporarily securing the metatarsophalangeal joint in the reduced position while the graft is attached.

10. The method of claim 9 wherein temporarily securing the metatarsophalangeal joint comprises attaching an instrument adjacent to the metatarsophalangeal joint with a first portion attached to the first metatarsus, a second portion attached to the proximal phalanx and a window aligned with the joint.

11. The method of claim 1 wherein the graft is attached to the first metatarsus and proximal phalanx.

12. The method of claim 11 wherein the graft is further attached to a sesamoid bone.

13. The method of claim 1 wherein the graft has at least three vertices.

14. The method of claim 13 wherein the graft is generally triangular and has a base dimension in the range of 11-35 mm and a height in the range of 9-29 mm.

15. The method of claim 13 wherein the graft is generally trapezoidal and has a first base dimension in the range of 11-35 mm, a second base dimension in the range of 4-14 mm, and a height in the range of 9-29 mm.

16. The method of claim 1 further comprising temporarily securing the metatarsophalangeal joint in the reduced position while the joint is stabilized.

17. A graft operable to stabilize a metatarsophalangeal joint, the graft comprising a generally planar structure having at least three vertices with a base dimension and a height dimension sized to span a metatarsophalangeal joint of a first ray of a human foot.

18. The graft of claim 17 wherein the base and height are sized to span an origin on a metatarsus and insertion on a proximal phalanx of a collateral ligament and the origin on the metatarsus and insertion on a sesamoid bone of a metatarsosesamoid ligament.

19. The graft of claim 17 wherein the graft is generally triangular and has a base dimension in the range of 11-35 mm and a height in the range of 9-29 mm.

20. The graft of claim 17 wherein the graft is generally trapezoidal and has a first base dimension in the range of 11-35 mm, a second base dimension in the range of 4-14 mm, and a height in the range of 9-29 mm.
